(19) United States
(12) Patent Application Publication
(10) Pub. No.: US 2026/0325503 A1
(43) Pub. Date: Oct. 22, 2026

(54) LONG-ACTING FORMULATIONS

(71) Applicant: JANSSEN PHARMACEUTICA NV, Beerse (BE)

(72) Inventors: Mukul Arun ASHTIKAR, Beerse (BE); Rudolf Josephus Dijmphna LEEMANS, Beerse (BE); An VAN CLEEMPOEL, Beerse (BE); Greet Lily MEURS, Beerse (BE)

(21) Appl. No.: 19/115,820

(22) PCT Filed: Sep. 27, 2023
(86) PCT No.: PCT/EP2023/076647

§ 371 Date: Mar. 27, 2025

(30) Foreign Application Priority Data
Sep. 28, 2022 (EP) 22198411.5

(51) Int. Cl. A61K 31/47; A61K 9/10; A61K 47/10

(52) U.S. Cl. CPC A61K 31/47; A61K 9/10; A61K 47/10

(57) ABSTRACT

This invention concerns pharmaceutical compositions for administration via intramuscular or subcutaneous injection, comprising micro-particles of the anti-TB compound bedaquiline, suspended in an aqueous pharmaceutically acceptable carrier, and the use of such pharmaceutical compositions in the treatment and prophylaxis of a pathogenic mycobacterial infection.

### FIELD OF THE INVENTION

This invention concerns pharmaceutical compositions for administration via intramuscular or subcutaneous injection, comprising micro- or nanoparticles of the ATP synthase inhibitor compound, bedaquiline (marketed as Sirturo®, where bedaquiline is in the form of its fumarate salt), suspended in an aqueous pharmaceutically acceptable carrier, and the use of such pharmaceutical compositions in the treatment of bacterial infections, e.g. tuberculosis and the like.

### BACKGROUND OF THE INVENTION

Bedaquiline is a known anti-tuberculosis drug used in various combinations. It may be formulated in the form of a pharmaceutically acceptable salt, such as in the form of bedaquiline fumarate, marketed as Sirturo®. It is thought to act as an ATP synthase inhibitor, possessing a selectivity index of more than 20000 for mycobacterial ATP synthase versus eukaryotic mitochondrial ATP synthase.

Bedaquiline has already been reported as being useful in the treatment of mycobacterial infections, as well as being useful in killing dormant, latent, persistent mycobacteria, in particular Mycobacterium tuberculosis, and can consequently be used to treat latent TB. Such use of bedaquiline has been described in several publications including international patent documents WO 2004/011436 and WO 2006/067048. It is also known that bedaquiline is bactericidal against mycobacterium leprae, for example as described in “Bacterial Activities of R207910 and other Antimicrobial Agents against Mycobacterium leprae in Mice”, Antimicrobial agents and Chemotherapy, April 2006, p 1558, and “The Diarylquinolone R207910 is Bactericidal against Mycobacterium leprae in mice and at Low Dose Administered Intermittently”, Antimicrobial agents and Chemotherapy, Sep. 2009, p 3989.

The goal of long-acting formulations can be to reduce drug burden. This is particularly useful for treatment regimens that may last several months.

The number and/or volume of dosage forms that need to be administered are commonly referred to as “pill burden”. A high pill burden is undesirable for many reasons, such as the frequency of intake, often combined with the inconvenience of having to swallow large dosage forms, as well as the need to store and transport a large number or volume of pills. A high pill burden increases the risk of patients not taking their entire dose, thereby failing to comply with the prescribed dosage regimen. As well as reducing the effectiveness of the treatment, this may also lead to the emergence of resistance (e.g. in the case of bedaquiline, bacterial resistance).

It would be attractive to provide therapy involving the administration of dosage forms at long time intervals such as one week or longer, or even one month or longer.

Various formulations are known in the art, including long-acting ones. For instance, micro- and nano-suspension technology is known for achieving long-acting formulations in the field of anti-HIV drugs, for instance as described in international patent applications WO 2007/147882 and WO 2012/140220. Further, nanoparticles known in the prior art have been described, for example, in EP-A-0 499 299. Such particles have an average particle size in the submicron range and consist of particles of a crystalline drug substance having a surface modifier adsorbed on their surface. Nanoparticles have also been used to formulate poorly water-soluble active ingredients.

Long-acting formulations of the anti-tuberculosis drug bedaquiline are also described in, for example, international patent applications WO 2019/012100, WO 2022/008643 and WO 2022/008645.

The importance of long-acting formulations relates to the intermittent administration of these micro- or nanoparticle formulations at time intervals of one week or longer that result in plasma levels that may be sufficient to suppress the growth of the mycobacterial infection. This allows for a reduced number of administrations thereby being beneficial in terms of pill burden and drug compliance of the patient. Micro- or nanoparticle formulations of bedaquiline therefore may be useful in the long-term treatment of mycobacterial infections (e.g. tuberculosis, including latent tuberculosis, and leprosy).

The intermittent administration of micro- or nanoparticle formulations of bedaquiline at time intervals of one week or longer furthermore results in plasma levels that may be sufficient to provide prevention against transmission of mycobacterial infection. Also in this instance, a reduced number of administrations is required, which again is advantageous in terms of pill burden and drug compliance of the individual at risk of being infected.

A challenge relating to the manufacture and suitability of such long-acting formulations relates to fact that they have to be sterilized (which is important for injectables, for instance if they are intended to be administered intravenously or subcutaneously). There are a number of different ways to sterilize such long-acting formulations, including by heat sterilization, aseptic manufacturing processes, autoclaving and gamma-radiation (γ-radiation). An example of some methods are described in e.g. US patents/applications U.S. Pat. Nos. 5,298,262, 5,346,702 and US 2010/255102. For heat sterilization and autoclaving, it is important to be able to select excipients (e.g. surface modifiers or surfactants) that are autoclavable, e.g. do not degrade. Further challenges arise after such sterilization, which are linked to desired stability of the long-acting formulation, undesired aggregation of particles of the active pharmaceutical ingredient (API) within that formulation and the desired re-suspendability of the formulation (after sterilization, e.g. autoclaving).

Thus, a challenge, is not only to produce long-acting formulations, but to find ones that are suitable for maintaining a certain level of stability for a sufficient period.

Now further alternative and/or improved long acting formulations are described, and the invention relates to such formulations.

### SUMMARY OF THE INVENTION

The present invention is concerned with a pharmaceutical composition for administration by intramuscular or subcutaneous injection, comprising a therapeutically effective amount of bedaquiline, or a pharmaceutically acceptable salt thereof, in the form of a suspension of microparticles, prepared by an aseptic manufacturing process, and comprising:
(a) bedaquiline, or a pharmaceutically acceptable salt thereof, in microparticle form, which is present, by weight based on the total volume of the composition, from 10% to 30% (w/v);
(b) a surface modifier, present from 5% to 15% (w/v), and which comprises at least 30% PEG4000 (or the like) and at least 30% poloxamer (such as poloxamer 338); and
(c) a pharmaceutically acceptable aqueous carrier,
and wherein such a composition may be referred to herein as “composition(s) of the invention”.

The composition of the invention is a suspension, by which we mean that the bedaquiline active ingredient is suspended in the pharmaceutically acceptable aqueous carrier.

The composition of the invention (i.e. the suspension) contains a surface modifier, which may be adsorbed onto the surface of the active ingredient bedaquiline.

In an embodiment, the present invention may therefore concern a pharmaceutical composition for administration by intramuscular or subcutaneous injection, comprising a therapeutically effective amount of bedaquiline, or a pharmaceutically acceptable salt thereof, in the form of a suspension of microparticles, prepared by an aseptic manufacturing process, and comprising:
(a) bedaquiline, or a pharmaceutically acceptable salt thereof, in microparticle form, having a surface modifier adsorbed to the surface thereof, and
(b) a pharmaceutically acceptable aqueous carrier; wherein the bedaquiline active ingredient is suspended,
wherein the bedaquiline (or pharmaceutically acceptable salt thereof) is present, by weight based on the total volume of the composition, from 10% to 30% (w/v) and the surface modifier present from 5% to 15% (w/v), and which comprises at least 30% PEG4000 (or the like) and at least 30% poloxamer (such as poloxamer 338).

The compositions of the invention are suspensions of microparticles, prepared by an aseptic manufacturing process, and as indicated the bedaquiline (or pharmaceutically acceptable salt thereof) and surface modifier (PEG4000 and poloxamer) are present in certain amounts; in certain further embodiments, they are present as following:
bedaquiline (or pharmaceutically acceptable salt thereof) is present from about 15% to 25% (w/v), for instance about 20% (w/v); and/or
the surface modifier is present from about 8% to 12% (w/v), which comprises the PEG4000 to poloxamer ratios described herein

The compositions of the invention have been optimized based on a number of parameters, and the optimized surface modifier quantities are as mentioned above; in certain further embodiments, they may also be present in the following quantities:
the surface modifier comprises between 30% and 70% PEG4000 (or the like) and hence the remainder consists of between 30% and 70% poloxamer;
the surface modifier comprises between 40% and 60% PEG4000, and between 40% and 60% poloxamer
the surface modifier comprises between 45% and 55% PEG4000 (e.g. about 50%), and between 45% and 50% poloxamer (e.g. about 50%)
the surface modifier comprises about 2 to 15% (e.g. 4 to 6%) PEG4000 (w/v), and about 2 to 15% (e.g. 4 to 6%) poloxamer (w/v)
the surface modifier comprises about 5% PEG4000 (w/v), and about 5% poloxamer (w/v)

The compositions of the invention are also indicated as containing a pharmaceutically acceptable carrier, which is described in more detail hereinafter.

The invention further concerns a method of treating a subject infected with pathogenic mycobacteria such as Mycobacterium tuberculosis, M. bovis, M. leprae, M avium and M. marinum. In an embodiment, the mycobacteria is Mycobacterium tuberculosis (including the latent or dormant form) or Mycobacterium leprae. The compositions of the invention may be particularly suitable for the treatment of Mycobacterium leprae and the latent or dormant form of Mycobacterium tuberculosis. This is because for treating these specific infections, a lower concentration of bedaquiline in the plasma may be effective against such infection, for instance as described in Antimicrobial Agents and Chemotherapy, September 2009, p. 3989-3991 by Robert Gelber, Koen Andries et al (the contents of which are hereby incorporated by reference, and wherein, essentially, it is reported that low and intermittent dosing with bedaquiline holds promise for leprosy patients; whereas minimal dose killing 99% of bacilli for M. tuberculosis is 30 mg/kg/wk, for M. lepra it is <5.0 mg/kg/wk, and hence dosing once a month may be as efficient as 5 days a week; other publications of the effect of bedaquiline on Mycobacterium leprae in mice include Antimicrobial Agents and Chemotherapy, April 2006, p. 1558-1560 by Baohong Ji, Koen Andries et al—the contents of which are also hereby incorporated by reference). Hence, the compositions of the invention may be particularly suitable in a method of treating a subject infected with Mycobacterium leprae or the latent/dormant form of Mycobacterium tuberculosis. Such methods of treating a subject infected with pathogenic mycobacteria comprise the administration, by intramuscular or subcutaneous injection, of a therapeutically effective amount of a pharmaceutical composition as specified above or hereinafter. Or, alternatively, the invention concerns the use of a pharmaceutical composition as specified above or hereinafter, for the manufacture of a medicament for treating pathogenic mycobacteria infection (or for using such medicament in a particular treatment regime as described herein). In one embodiment, the composition is for the long-term treatment of pathogenic mycobacteria infection. In an embodiment, the pathogenic mycobacterial infection may such as described above or hereinafter, such as an infection that requires long-term treatment (in a further embodiment, an infection that further may be treated at relatively low plasma concentration levels of bedaquiline or its active metabolite, for instance latent/dormant Mycobacterium tuberculosis or, in a particular embodiment, Mycobacterium leprae).

In another aspect, there is provided a method for the long term treatment of a subject infected with pathogenic mycobacteria such as Mycobacterium tuberculosis, M. bovis, M. leprae, M. avium and M. marinum, said method comprising the administration of an effective amount of a pharmaceutical composition as specified above or hereinafter, for administration by intramuscular or subcutaneous injection; wherein the composition is administered or is to be administered intermittently at a time interval that is in the range of one week to one year, or one week to two years. Or, alternatively, the invention concerns the use of a pharmaceutical composition as specified above or hereinafter, for the manufacture of a medicament for the long term treatment of a subject infected with pathogenic mycobacteria such as Mycobacterium tuberculosis, M. bovis, M. leprae, M. avium and M. marinum, for administration by intramuscular or subcutaneous injection, wherein the composition is administered or is to be administered intermittently at a time interval that is in the range of one week to one year, or one week to two years. Hence, it will be understood that the term “long term treatment” refers to treatment where one dose or one administration (e.g. by intramuscular or subcutaneous injection) will have a persistent therapeutic effect over a time period, as described herein, for instance a persistent therapeutic effect over several hours, weeks or months (e.g. in an embodiment, over a period of at least or up to one month, three months or six months); see examples. Put another way, long term treatment may refer to, where there is more than one dose/administration, the long period of time (as described herein) between the doses/administrations, i.e. the intervals are a long period of time as described herein.

In another aspect, there is provided a method for the long term treatment of a subject infected with pathogenic mycobacteria (e.g. of any of the types as described here), as described herein (e.g. above) wherein one dose or administration (e.g. of the amount described herein, e.g. hereinafter) is provided/required (and has a persistent effect, e.g. over a time period described herein). In another aspect, there is provided such a long term treatment regime, where two such doses or administrations are provided/required, which doses/administrations are given at intervals, wherein the interval time period is that as described herein, e.g. a period of at least or up to one month, three months or six months—for instance for a period of time in which persistent therapeutic effect lasts). In a further embodiment, there is provided such a long term treatment regime, in which three such doses or administrations are provided/required at such intervals as herein described. In yet a further embodiment, there is provided a long term treatment regime as herein described but which is preceded with a lead-in treatment phase (that is not a long term treatment regime, e.g. a once-daily administration course, lasting for one week, two weeks, three weeks or one month).

The invention further concerns a method for the prevention of a pathogenic mycobacterial infection in a subject at risk of being infected by a pathogenic mycobacterial infection, said method comprising administering an amount, effective in preventing a pathogenic mycobacterial infection, of a pharmaceutical composition as specified above or as further specified hereinafter, to said subject. Or alternatively, the invention concerns the use of a pharmaceutical composition as specified above or as further specified hereinafter for the manufacture of a medicament for the prevention of a pathogenic mycobacterial infection in a subject at risk of being infected by a pathogenic mycobacterial infection.

In another aspect the invention relates to a method for the long term prevention of a pathogenic mycobacterial infection in a subject at risk of being infected by a pathogenic mycobacterial infection, said method comprising administering to said subject an effective amount of a pharmaceutical composition as specified above or as further specified hereinafter, wherein the composition is administered or is to be administered intermittently at a time interval that is in the range of one week to one year, or one week to two years.

The present invention furthermore relates to the use of a pharmaceutical composition as specified above or as further specified hereinafter, for the manufacture of a medicament for the long term prevention for the long term prevention of a pathogenic mycobacterial infection in a subject at risk of being infected by a pathogenic mycobacterial infection, wherein the composition is administered or is to be administered intermittently at a time interval that is in the range of one week to one year or one week to two years.

In one embodiment the invention concerns a use or a method as specified herein, wherein the pharmaceutical composition is administered or is to be administered at a time interval that is in the range of one week to one month, or in the range of one month to three months, or in the range of three months to six months, or in the range of six months to twelve months, or in the range of 12 months to 24 months.

In another embodiment the invention concerns a use or a method as specified herein, wherein the pharmaceutical composition is administered or is to be administered once every two weeks, or once every month, or once every three months.

Further pharmaceutical compositions, methods of treatment or prevention, as well as uses for the manufacture of medicaments based on these compositions will be described hereinafter and are meant to be part of the present invention.

### DETAILED DESCRIPTION OF THE INVENTION

The compound used in the invention is the compound TMC207, also referred to as bedaquiline.

Bedaquiline can be used in its non-salt form or as a suitable pharmaceutically acceptable salt form, such as an acid addition salt form or base addition salt form. In an embodiment, bedaquiline is in its non-salt form in compositions of the invention.

The pharmaceutically acceptable acid addition salts are defined to comprise the therapeutically active non-toxic acid addition salt forms which bedaquiline is able to form. Said acid addition salts can be obtained by treating the free form of bedaquiline with appropriate acids, for example inorganic acids, for example hydrohalic acid, in particular hydrochloric acid, hydrobromic acid, sulfuric acid, nitric acid and phosphoric acid; organic acids, for example acetic acid, hydroxyacetic acid, propanoic acid, lactic acid, pyruvic acid, oxalic acid, malonic acid, succinic acid, maleic acid, fumaric acid, malic acid, tartaric acid, citric acid, methanesulfonic acid, ethanesulfonic acid, benzenesulfonic acid, p-toluenesulfonic acid, cyclamic acid, salicyclic acid, p-aminosalicylic acid and pamoic acid. In particular, the fumarate salt is considered, given that this is the form employed in the already-marketed product Sirturo®.

Possible therapeutically active non-toxic base addition salt forms may be prepared by treatment with appropriate organic and inorganic bases. Appropriate base salts forms comprise, for example, the ammonium salts, the alkaline and earth alkaline metal salts, in particular lithium, sodium, potassium, magnesium and calcium salts, salts with organic bases, e.g. the benzathine, N-methyl-D-glucamine, hybramine salts, and salts with amino acids, for example arginine and lysine.

Conversely, said acid or base addition salt forms can be converted into the free forms by treatment with an appropriate base or acid.

The term addition salt as used in the framework of this application also comprises the solvates which bedaquiline as well as the salts thereof, are able to form. Such solvates are, for example, hydrates and alcoholates.

Whenever reference to bedaquiline (or TMC207) is employed herein, we refer to the single stereoisomeric form that is employed in the marketed product Sirturo®, and which is disclosed in WO2004/011436 as an antimycobacterial agent.

It has been found that the physico-chemical properties of bedaquiline allow for the manufacture of micro-particle suspensions that have unique pharmacokinetic properties in that they can be used for the long term treatment of a pathogenic mycobacterial infection as well as in the long term prevention of a pathogenic mycobacterial infection and to this purpose only a limited number of drug administrations is required. This is beneficial in terms of pill-burden as well as patient compliance with the prescribed dose regimen.

As used herein the term “treatment of a pathogenic mycobacterial infection” relates to the treatment of a subject being infected with a pathogenic mycobacterial infection.

The term “prevention of a pathogenic mycobacterial infection” relates to the prevention or avoidance of a subject becoming infected with a pathogenic mycobacterial infection. The source of infection can be various, for instance a material containing a pathogenic mycobacterial infection.

The terms “therapeutically effective amount”, “an amount, effective in preventing a pathogenic mycobacterial infection”, and similar terms, refer to amounts, or concentrations, of the compositions of the invention (or amounts/concentrations of active ingredient bedaquiline within such compositions) that result in efficacious plasma levels. With “efficacious plasma levels” it is meant those plasma levels of bedaquiline that provide effective treatment or effective prevention of a pathogenic mycobacterial infection. This is because amount/dose/administration given may be linked to the desired exposure levels or desired plasma levels for the effective treatment/prevention, for instance as described herein (see e.g. the examples).

The term “subject” in particular relates to a human being.

The term “micro- or nanoparticles” refers to particles in the micrometer or nanometer range. The size of the particles should be below a maximum size above which administration by subcutaneous or intramuscular injection becomes impaired or is even no longer possible. Said maximum size depends for example on the limitations imposed by the needle diameter or by adverse reactions of the body to large particles, or both. In one embodiment, the pharmaceutical compositions of the invention comprise bedaquiline in microparticle form.

The average effective particle size of the micro-particles of the present invention may be below about 50 μm, or below about 20 μm, or below about 10 μm, or below about 1000 nm, or below about 500 nm, or below about 400 nm, or below about 300 nm, or below about 200 nm. For instance, both after preparation and after a period of time of up to 3 months (e.g. when stored at temperatures of about 5° C., 25° C. and 40° C.) generally:
the micro-suspensions (or compositions of the invention) may have, in an embodiment, a D90 of between about 3 and 10 μm (e.g. between about 4 to 7 μm, e.g. about 5 or 6 μm); a D50 of between about 2 and 4 μm (e.g. between about 2 and 3 μm, e.g. about 2.5 μm) and a D10 of between about 0.3 and 1.5 μm (e.g. between about 0.5 and 1 μm, e.g. about 0.7 or 0.8 μm)—all of these D10/D50/D90 values may refer to those after preparation (i.e. at T0, or time zero) or may also refer to such values after a certain period of time (e.g. 1 month/1M, 3M, 6M) where they are stored at a certain temperature (e.g. 30° C., 40° C., 50° C. or 5° C.).

In an embodiment, the micro-particles are employed, wherein the average effective particle size, as measured by D10, D50 and/or D90 (in an embodiment as measured by D50) is below about 50 μm, or below about 20 μm, and above about 0.1 μm (100 nm). In an embodiment the range for such micro-particles employed in the compositions of the invention is between about 20 μm and about 0.1 μm (in a further embodiment between about 15 μm, and above about 0.2 μm and in a further embodiment between about 10 μm, and above 0.2 μm, for instance between about 10 μm, and above 0.2 μm (see also values above and hereinafter, where specific values of D10/D50/D90 are measured). The foregoing values refer to measurements after preparation. They may also, however, in an embodiment, refer to measurements after a period of time up to 3 months (e.g. after 5 days, one week, two weeks, one month, two months or three months) and stored at various temperatures (e.g. at temperatures of about 5° C., 25° C., 30° C., 40° C. and 50° C.). An advantage of the micro-suspensions of the invention is that the average effective particle size, as measured herein by D10/D50/D90 values, and also referred to as particle size distribution (PSD) does not significantly change, but rather the D10/D50/D90 values remain relatively stable—by which we mean there is a less than 25%, for instance a less than 15% deviation within the values.

As used herein, the term average effective particle size has its conventional meaning as known to the person skilled in the art and can be measured by art-known particle size measuring techniques such as, for example, sedimentation field flow fractionation, photon correlation spectroscopy, laser diffraction or disk centrifugation. The average effective particle sizes mentioned herein may be related to volume distributions of the particles. In that instance, by “an effective average particle size of less than about 50 μm” it is meant that at least 50% of the volume of the particles has a particle size of less than the effective average of 50 μm, and the same applies to the other effective particle sizes mentioned. In a similar manner, the average effective particle sizes may be related to weight distributions of the particles but usually this will result in the same or about the same value for the average effective particle size.

The pharmaceutical compositions of the present invention provide release of the active ingredient bedaquiline over a prolonged period of time and therefore they can also be referred to as sustained or delayed release compositions. After administration, the compositions of the invention stay in the body and steadily release bedaquiline, keeping such levels of this active ingredient in the patient's system for a prolonged period of time, thereby providing, during said period, the appropriate treatment or prevention of a pathogenic mycobacterial infection. Because of the fact that the pharmaceutical compositions of the invention stay in the body and steadily release bedaquiline (and its active metabolite, referred to as M2 herein; see hereinafter, the methyl-substituted metabolite), they can be referred to as pharmaceutical compositions suitable as long-acting (or depot) formulations.

As used herein with the term “prolonged period of time”, there is meant a term (or time period) that may be in the range of one week up to one year or up to two years, or a term in the range of one to two weeks, or two to three weeks, or three to four weeks, or a term in the range of one to two months, or two to three months, or three to four months, or three to six months, or six months to 12 months, or 12 months to 24 months, or a term that is in the range of several days, e.g. 7, 10 or 12 days, or several weeks, e.g. 2, 3 or 4 weeks, or one month, or several months, e.g. 2, 3, 4, 5 or six months or even longer, e.g. 7, 8, 9 or 12 months.

The pharmaceutical compositions of this invention may be applied in the long-term treatment or the long-term prevention of a pathogenic mycobacterial infection, or with other words they may be used in the treatment of a pathogenic mycobacterial infection, or in the prevention of a pathogenic mycobacterial infection, during a prolonged period of time. The compositions of the invention are effective in the treatment or prevention of a pathogenic mycobacterial infection for a prolonged period of time, for example for at least about one week or longer, or for about 1 month or longer. By the expression “effective for at least about one week or longer”, one means that the plasma level of the active ingredient, bedaquiline (and/or its active metabolite M2), should be above a threshold value. In case of therapeutic application said threshold value is the lowest plasma level at which bedaquiline (and/or its active metabolite M2) provides effective treatment of a pathogenic mycobacterial infection. In case of application in the prevention of a pathogenic mycobacterial infection said threshold value is the lowest plasma level at which bedaquiline (and/or its active metabolite M2) is effective in preventing transmission of a pathogenic mycobacterial infection.

With “long term” for example as used in relation to “long term prevention of a pathogenic mycobacterial infection” or “long term treatment of a pathogenic mycobacterial infection”, or similar terminology, there are meant terms that may be in the range of one week up to one year or up to two years, or longer, such as five or 10 years. In particular in the case of treatment of a pathogenic mycobacterial infection, such terms will be long, in the order of one to several months, one year or longer. Such terms may also be relatively short, in particular in the case of prevention. Shorter terms are those of several days, e.g. 7, 10 or 12 days, or several weeks, e.g. 2, 3 or 4 weeks, or one month, or several months, e.g. 2, 3, 4, 5 or six months or even longer, e.g. 7, 8, 9 or 12 months. In one embodiment the methods and uses in accordance with the present invention are for the prevention of a pathogenic mycobacterial infection during one month, or several months, e.g. 2, 3, 4, 5 or six months or even longer, e.g. 7, 8, 9 or 12 months.

The pharmaceutical compositions of the present invention can be administered at various time intervals. When used in the prevention of a pathogenic mycobacterial infection, the pharmaceutical compositions of this invention can be administered only once or a limited number of times such as twice, three, four, five or six times, or more. This may be recommendable where prevention is required during a limited period of time, such as the period during which there is a risk of infection.

The pharmaceutical compositions of the present invention can be administered at the time intervals mentioned above, such as at a time interval that is in the range of one week to one month, or in the range of one month to three months, or in the range of three months to six months, or in the range of six months to twelve months. In one embodiment, the pharmaceutical composition can be administered once every two weeks, or once every month, or once every three months. In another embodiment the time interval is in the range of one to two weeks, or two to three weeks, or three to four weeks, or the time interval is in the range of one to two months, or two to three months, or three to four months, or three to six months, or six months to 12 months, or 12 months to 24 months. The time interval may be at least one week, but may also be several weeks, e.g. 2, 3, 4, 5 or 6 weeks, or at time intervals of one month, or of several months, e.g. 2, 3, 4, 5 or 6 months or even longer, e.g. 7, 8, 9 or 12 months. In one embodiment, the pharmaceutical compositions of the present invention are administered at a time interval of one, two or three months. These longer periods between each administration of the pharmaceutical compositions of the invention provide further improvements in terms of pill burden and compliance. To further improve compliance, patients can be instructed to take their medication at a certain day of the week, where the composition is administered on a weekly schedule, or at a certain day of the month in case of a monthly schedule.

The length of the time intervals between each administration of a composition of the present invention may vary. For example said time intervals may be selected in function of the plasma levels. The intervals may be shorter where the plasma levels of bedaquiline (and/or its active metabolite M2) are deemed too low, e.g. when these approach the minimum plasma level specified hereinafter. The intervals may be longer where the plasma levels of bedaquiline (and/or its active metabolite M2) are deemed too high. In one embodiment, the compositions of the invention are administered at equal time intervals. The compositions may be administered without any interjacent additional administrations, or with other words, the compositions may be administered at particular points in time separated from one another by a time period of varying or equal length, e.g. a time period of at least one week, or any other time period specified herein, during which no further bedaquiline is administered. Having time intervals of the same length has the advantage that the administration schedule is simple, e.g. administration takes place at the same day in the week, or the same day in the month. Such administration schedule therefore involves limited “pill burden” thereby contributing beneficially to the patient's compliance to the prescribed dosing regimen.

The concentration (or “C”) of bedaquiline (and/or its active metabolite M2) in the plasma of a subject treated therewith is generally expressed as mass per unit volume, typically nanograms per milliliter (ng/ml). For convenience, this concentration may be referred to herein as “plasma drug concentration” or “plasma concentration”.

The dose (or amount) of bedaquiline administered, depends on the amount of bedaquiline in the pharmaceutical compositions of the invention, or on the amount of a given composition that is administered. Where higher plasma levels are desired, either or both of a composition of higher bedaquiline concentration, or more of a given composition, may be administered. This applies vice versa if lower plasma levels are desired. Also a combination of varying time intervals and varying dosing may be selected to attain certain desired plasma levels.

The dose (or amount) of bedaquiline administered also depends on the frequency of the administrations (i.e. the time interval between each administration). Usually, the dose will be higher where administrations are less frequent. All these parameters can be used to direct the plasma levels to desired values.

The dosing regimen also depends on whether prevention or treatment of the pathogenic mycobacterial infection is envisaged. In case of therapy, the dose of bedaquiline administered or the frequency of dosing, or both, are selected so that the plasma concentration of bedaquiline is kept above a minimum plasma level. The term “minimum plasma level” (or Cₘᵢₙ) in this context refers to the plasma level of bedaquiline (and/or its active metabolite M2) that provides effective treatment of the pathogenic mycobacterial infection. In particular, the plasma level of bedaquiline (and/or its active metabolite M2) is kept at a level above a minimum plasma level of about 10 ng/ml, or above about 15 ng/ml, or above about 20 ng/ml, or above about 40 ng/ml. The plasma level of bedaquiline (and/or its active metabolite M2) may be kept above a minimum plasma level that is higher, for example above about 50 ng/ml, or above about 90 ng/ml, or above about 270 ng/ml, or above about 540 ng/ml. In one embodiment, the plasma level of bedaquiline (and/or its active metabolite M2) is kept above a level of about 13.5 ng/ml, or is kept above a level of about 20 ng/ml. Or the plasma level of bedaquiline (and/or its active metabolite M2) may be kept within certain ranges, in particular ranges starting from a minimum plasma level selected from those mentioned above and ending at a higher plasma levels selected from those mentioned above and selected from 500 ng/ml and 1000 ng/ml (e.g. from 10 to 15, 10 to 20, 10 to 40, etc., or from 15 to 20, or 15 to 40, or 15 to 90, etc., or 20 to 40, 20 to 90, or 20 to 270, etc., or 40 to 90, 40 to 270, or 40-540, etc., each time from about the indicated value in ng/ml to about the indicated value in ng/ml). In one embodiment said range is from about 10 to about 20, from about 20 to about 90, from 90 to 270, from 270 to 540, from 540 to 1000, each time from about the indicated value in ng/ml to about the indicated value in ng/ml.

The plasma levels of bedaquiline (and/or its active metabolite M2) should be kept above the above-mentioned minimum plasma levels because at lower levels the bacteria may no longer be sufficiently suppressed so that it can multiply with the additional risk of the emergence of mutations.

In the instance of prevention, the term “minimum plasma level” (or Cₘᵢₙ) refers to the lowest plasma level of bedaquiline (and/or its active metabolite M2) that provides effective treatment/prevention of infection.

In particular, in the instance of prevention, the plasma level of bedaquiline (and/or its active metabolite M2) can be kept at a level above a minimum plasma level mentioned above in relation to therapy. However in prevention the plasma level of bedaquiline (and/or its active metabolite M2) can be kept at a lower level, for example at a level above about 4 ng/ml, or about 5 ng/ml, or about 8 ng/ml. The plasma levels of bedaquiline (and/or its active metabolite M2) should preferably be kept above these minimum plasma levels because at lower levels the drug may no longer be effective thereby increasing the risk of transmission of infection. Plasma levels of bedaquiline (and/or its active metabolite M2) may be kept at somewhat higher levels to have a safety margin. Such higher levels start from about 50 ng/ml or more. The plasma level of bedaquiline (and/or its active metabolite M2) can be kept at a level that is in the ranges mentioned above in relation to therapy, but where the lower limits include the plasma levels of about 4 ng/ml, or about 5 ng/ml, or about 8 ng/ml.

An advantage of bedaquiline (and/or its active metabolite M2) is that it may be used up to relatively high plasma levels without any significant side effects. The plasma concentrations of bedaquiline (and/or its active metabolite M2) may reach relatively high levels, but as with any drug should not exceed a maximum plasma level (or Cₘₐₓ), which is the plasma level where bedaquiline (and/or its active metabolite M2) causes significant side effects. Additionally, compound-release from the tissue should also be taken into account, which is not counted for within plasma levels. As used herein, the term “significant side effects” means that the side effects are present in a relevant patient population to an extend that the side effects affect the patients' normal functioning. In an embodiment, the amount and the frequency of administrations of bedaquiline (and/or its active metabolite M2) to be administered are selected such that the plasma concentrations are kept during a long term at a level comprised between a maximum plasma level (or Cₘₐₓ as specified above) and a minimum plasma level (or Cₘᵢₙ as specified above).

In certain instances it may be desirable to keep the plasma levels of bedaquiline (and/or its active metabolite M2) at relatively low levels, e.g. as close as possible to the minimum plasma levels specified herein. This will allow reducing the frequency of the administrations and/or the quantity of bedaquiline (and/or its active metabolite M2) administered with each administration. It will also allow avoiding undesirable side effects, which will contribute to the acceptance of the dosage forms in most of the targeted population groups who are healthy people at risk of being infected and therefore are less inclined to tolerate side effects. The plasma levels of bedaquiline (and/or its active metabolite M2) may be kept at relatively low levels in the instance of prevention. One embodiment concerns uses or methods for prevention of infection, as specified above or hereinafter, wherein the minimum plasma level of bedaquiline (and/or its active metabolite M2) is as specified herein and the maximum plasma level is about equal to the lowest plasma level that causes the active ingredient to act therapeutically, also as specified herein.

In other embodiments, the plasma level of bedaquiline (and/or its active metabolite M2) is kept at a level below a lower maximum plasma level of about 10 ng/ml, more in particular about 15 ng/ml, further in particular about 20 ng/ml, still more in particular about 40 ng/ml. In a particular embodiment, the plasma level of bedaquiline (and/or its active metabolite M2) is kept below a level of about 13.5 ng/ml. In one embodiment, the plasma level of bedaquiline (and/or its active metabolite M2) is kept in an interval of the lower maximum blood level specified above, and the minimum plasma levels mentioned in relation to prevention. For example the plasma levels of bedaquiline (and/or its active metabolite M2) are kept below about 10 ng/ml and above a minimum level of about 4 ng/ml.

In other instances it may be desirable to keep the plasma levels of bedaquiline (and/or its active metabolite M2) at relatively higher levels, for example where there is a high risk of infection and more frequent and/or higher doses are not an issue. In these instances the minimum plasma level may be equal to the lowest plasma level of bedaquiline (and/or its active metabolite M2) that provides effective treatment of a pathogenic mycobacterial infection, such as the specific levels mentioned herein.

In the instance of prevention, the dose to be administered should be calculated on a basis of about 0.2 mg/day to about 50 mg/day, or 0.5 mg/day to about 50 mg/day, or of about 1 mg/day to about 10 mg/day, or about 2 mg/day to about 5 mg/day, e.g. about 3 mg/day. This corresponds to a weekly dose of about 1.5 mg to about 350 mg, in particular of about 3.5 mg to about 350 mg, in particular of about 7 mg to about 70 mg, or about 14 mg to about 35 mg, e.g. about 35 mg, or to a monthly dose of from 6 mg to about 3000 mg, in particular about 15 mg to about 1,500 mg, more in particular of about 30 mg to about 300 mg, or about 60 mg to about 150 mg, e.g. about 150 mg. Doses for other dosing regimens can readily be calculated by multiplying the daily dose with the number of days between each administration.

In the instance of therapy, the dose to be administered should be somewhat higher and should be calculated on a basis of about 1 mg/day to about 150 mg/day, or of about 2 mg/day to about 100 mg/day, or of about 5 mg/day to about 50 mg/day, or about 10 mg/day to about 25 mg/day, e.g. about 15 mg/day. The corresponding weekly or monthly doses can be calculated as set forth above. For applications in prevention, the doses may be lower although the same dosing as for therapeutic applications may be used. In an embodiment, the dose/administration is given at monthly intervals or three-monthly or six-monthly intervals, with the total treatment duration being three, six or 12 months. In the instances where the dose/administration is monthly, three monthly or six-monthly, in an embodiment, the dose given (e.g. in human subjects) is calculated on the basis of a 400 mg daily dose given for 2 weeks. Hence, the total amount of bedaquiline given per dose may be about 5600 mg (e.g. in the range of 3000 and 8000 mg), but it may be up to one fifth of such an amount (e.g. in the range of 500 and 2000 mg, e.g. between about 1000 and 1500 mg).

In another embodiment, in the case of prevention or in particular therapy, the doses may also be expressed in mg/kg. For instance, in the examples, certain doses may be administered based on weight (of e.g. the mammal, and as shown in the examples here, in mouse) and hence doses between 1 mg/kg and 1000 mg/kg may be employed (e.g. 40 mg/kg, 80 mg/kg, 160 mg/kg, 320 mg/kg or 480 mg/kg may be employed) and such doses may remain effective for a period of 4 weeks, 8 weeks or 12 weeks (for example as shown in the examples). For instance, one dose may be taken every 4 weeks (effectively seen as a 12 week treatment regimen, i.e. three doses in total) or one single dose may be taken, which effectively provides sufficient treatment (e.g. as defined by reduction in CFUs, see examples) as may be evidenced by monitoring over a 12 week period. Hence, in an aspect, in order to treat the bacterial infection one dose may be taken (e.g. between 1 mg/kg and 1000 mg/kg, for instance between 2 mg/kg and 500 mg/kg) or one such dose may be taken every 4 weeks (e.g. two or three such doses may be taken). Such dose depends on the bacterial infection to be treated. For instance, in the treatment of latent tuberculosis or leprosy, lower doses may be required (compared to e.g. multi-drug resistant tuberculosis) given that a lower amount of bedaquiline is required to control the bacteria.

It has been found that, once administered, the plasma levels of bedaquiline (and/or its active metabolite M2) are more or less stable, i.e. they fluctuate within limited margins. The plasma levels have been found to approach more or less a steady state mode or to approximate more or less a zero order release rate during a prolonged period of time. By “steady state” is meant the condition in which the amount of drug present in the plasma of a subject stays at more or less the same level over a prolonged period of time. The plasma levels of bedaquiline (and/or its active metabolite M2) generally do not show any drops below the minimum plasma level at which the drug is effective. The term “stays at more or less the same level” does not exclude that there can be small fluctuations of the plasma concentrations within an acceptable range, e.g. fluctuations within a range of about ±30%, or about ±20%, or about ±10%, or about ±10%.

In some instances there may be an initial plasma concentration peak after administration, after which the plasma levels achieve a “steady-state”, as mentioned hereinafter.

The compositions of the invention show good local tolerance and ease of administration. Good local tolerance relates to minimal irritation and inflammation at the site of injection; ease of administration refers to the size of needle and length of time required to administer a dose of a particular drug formulation. In addition, the compositions of the invention show good stability and have an acceptable shelf life.

The micro-particles of the present invention have a surface modifier adsorbed on the surface thereof. The function of the surface modifier is to act as a wetting agent as well as a stabilizer of the colloidial suspension.

In one embodiment, the micro-particles in the compositions of the invention mainly comprise crystalline bedaquiline or a salt thereof, and a surface modifier, the combined amount of which may at least comprise about 50%, or at least about 80%, or at least about 90%, or at least about 95%, or at least about 99% of the micro-particles. As indicated herein, in an embodiment, bedaquiline is in its non-salt form (or in its “free form”) and in a further embodiment it is in a crystalline non-salt (or free) form. In this respect, as mentioned herein, bedaquiline may be prepared as such using the procedures described in international patent application WO 2004/011436 (or in WO 2006/125769, which describes an optical resolution with a chiral reagent). Following such procedure, the bedaquiline is obtained by precipitation from toluene/ethanol and it is indicated that the product crystallises. Such form of bedaquiline may be used in the preparation of the compositions of the invention and, further, such form may be a single crystalline polymorph with the following characterising features:
(i) a melting endotherm at 181.5° C. (endotherm onset) and DSC curve showing melting of the product at about 182.5° C. (immediately followed by decomposition; measured by differential scanning calorimetry (DSC) by transfer of about 3 mg of compound into a standard aluminum TA-Instrument sample pan, sample pan closed with the appropriate coer and DSC curve recorded on a TA-Instruments Q2000 MTDSC equipped with a RCS cooling unit using the following parameters—initial temperature 25° C.; heating range 10° C./min; final temperature 300° C., nitrogen flow 50 ml/min);
(ii) infrared (IR) spectrum peaks at inter alia about 1600 cm⁻¹, about 1450 cm⁻¹, about 1400 cm⁻¹, about 1340 cm⁻¹, and about 1250 cm⁻¹ (where a sample is analysed using a suitable microATR accessory deploying 32 scans, 1 cm⁻¹ resolution, Thermo Nexus 670 FTIR spectrometer, a DTGS with KBr windows detector, Ge on KBr beamsplitter and a micro ATR accessory (Harrick Split Pea with Si crystal); and/or
(iii) X-ray powder diffraction (XRPD) with characteristic peaks at about 11.25° 2-Theta, about 18° 2-Theta, about 18.5° 2-Theta, about 19° 2-Theta, about 20.25° 2-Theta, about 21.25° 2-Theta, about 22.25° 2-Theta, about 24.5° 2-Theta and about 27° 2-Theta, showing diffraction peaks without the presence of a halo indicating crystallinity of the product (where the analysis was carried out on a PANalytical (Philips) X'PertPRO MPD diffractometer, and the instrument is equipped with a Cu LFF X-ray tube and the compound was spread on a zero background sample holder; the Instrument Parameters were: generator voltage—45 kV; generator amperage—40 mA; geometry—Bragg-Brentano; stage—spinner stage; scan mode—continuous; scan range 3 to 50° 2θ; step size 0.02°/step; counting time 30 sec/step; spinner revolution time—1 sec; radiation type CuKα).

Hence, in an embodiment, the bedaquiline employed in a process to prepare compositions of the invention (i.e. before conversion to micro-particles) is a crystalline form (e.g. of the specific form characterised above). In a further embodiment of the invention, the bedaquiline employed in the compositions of the invention (i.e. after conversion to micro-particles, for instance by milling) is also in a crystalline form (e.g. of the specific form characterised above). It is an advantage that the crystalline form that is part of the suspension/formulation of the invention does not substantially change when stored for certain periods of time at certain temperatures (for instances as described below).

Compositions of the invention are as described herein, wherein it is already specified that the bedaquiline is present in a certain amount (w/v), and it is already specified that the surface modifier contains PEG4000 (or the like) and poloxamer (such as poloxamer 338), where the overall amount of surface modifier is specified, and the ratios of PEG4000 to poloxamer (or the amounts in w/v) are also specified in certain ratios. Regarding the surface modifier, while small or trace amounts of other surface modifiers (as defined herein) may be present, such that they do not significantly alter the properties (e.g. chemical and/or physical properties) of the compositions, in an aspect of the invention, the surface modifier consists essentially of (e.g. consists of) only PEG4000 (or the like) and poloxamer, in any of the quantities or ratios specified herein.

PEG4000, or, polyethylene glycol 4000, is a known high-molecular weight polymer where the 4000 refers to the approximate average molecular weight in daltons. PEG4000 is commercially available from sources such as Sigma-Aldrich and hence why it is used as such. However, embraced within the scope of the invention (e.g. when the term “PEG4000” or “PEG4000, or the like” is used) are other high-molecular weight polyethylene glycols, for instance those above 1000 and up to 8000 (e.g. PEG1000 to PEG8000, for instance PEG2000 to PEG6000), even though in a particular embodiment the PEG group when referred to herein in the context of the invention is PEG3000 to PEG5000 (e.g. PEG3500 to PEG4500). As indicated herein, the number next to the PEG represents average molecule weight in daltons, as it is understood that most PEGs include molecules with a distribution of molecular weights, i.e. they are polydisperse.

The poloxamer used in the compositions of the invention may include poloxamers such as Pluronic™ F68, F108 and F127 which are block copolymers of ethylene oxide and propylene oxide; in an embodiment, the poloxamer used in the compositions of the invention is poloxamer 338 (which corresponds to Pluronica F108). Pluronica F108 corresponds to poloxamer 338 and is the polyoxyethylene, polyoxypropylene block copolymer that conforms generally to the formula HO—[CH₂CH₂O]ₓ—[CH(CH₃)CH₂O]_{y}—[CH₂CH₂O]_{z}—H in which the average values of x, y and z are respectively 128, 54 and 128.

Other commercial names of poloxamer 338 are Hodag Nonionics 1108-F and Synperonic™ PE/F108.

Suitable surface modifiers that may also be present in small or trace quantities (in addition to the essential PEG4000, or the like, and the poloxamer) can be selected from known organic and inorganic pharmaceutical excipients, including various polymers, low molecular weight oligomers, natural products and surfactants. Particular surface modifiers include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropylmethylcellulose, magnesium aluminate silicate, polyvinyl alcohol (PVA); tyloxapol; Vitamin E-TPGS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlace™ 20, 40, 60 and 80); polyethylene glycols (such as those sold under the tradename Carbowax™ 3550 and 934); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination.

Particular surface modifiers that may be present in small or trace amounts are selected from α-tocopheryl polyethylene glycol succinates, polyoxyethylene sorbitan fatty acid esters, and salts of negatively charged phospholipids or the acid form thereof. More in particular the surface modifiers are selected from Vitamin E TPGS, Tween™ 80, and Lipoid™ EPG (and, in a particular embodiment, it is Vitamin E TPGS). Others include a polyoxyethylene sorbitan fatty acid ester and a phosphatidyl glycerol salt (in particular egg phosphatidyl glycerol sodium).

The optimal relative amount of bedaquiline in relation to the surface modifier depends on the surface modifier selected, the specific surface area of the bedaquiline suspension which is determined by the average effective particle size and the bedaquiline concentration, the critical micelle concentration of the surface modifier if it forms micelles, etc. The relative amount (w/w) of bedaquiline to the surface modifier preferably is in the range of about 1:1 to about 10:1, in particular in the range of 1.5:1 to about 5:1, e.g. about 2:1.

The compositions of the invention may need to be sterile so that they can be administered to patients. Achieving sterile compositions may be done in a number of ways, including manufacturing such compositions in a sterile process or environment, for instance following an aseptic manufacturing process. Alternatives include heat sterilization, autoclaving and gamma irradiation, which serve as sterilization steps that can achieve the desired outcome. As specified, the compositions of the invention herein are prepared following an aseptic manufacturing process (e.g. ensuring a sterile process, equipment and environment); in a further embodiment, the compositions of the invention are prepared following an aseptic manufacturing process, and wherein the active pharmaceutical ingredient (API), i.e. the bedaquiline (or salt thereof) is separately γ-irradiated. The compositions of the invention, due to the nature of the composition (including its method of manufacture) may as a result have different chemical and/or physical properties—these are outlined in the data presented, and hence can be seen as advantageous properties. Re-suspendability of the compositions of the invention is also an important aspect, and indeed such compositions were quickly re-suspendable (e.g. through gentle swirling).

The particles of this invention can be prepared by means of micronization/particle size reduction by mechanical means and by controlled precipitation from a supersaturated solution, or by using supercritical fluids such as in the GAS technique (“gas anti-solvent”), or any combination of such techniques. In one embodiment a method is used comprising the steps of dispersing bedaquiline in a liquid dispersion medium and applying mechanical means in the presence of grinding media to reduce the particle size of bedaquiline to an average effective particle size of less than about 50 μm, in particular less than about 1,000 nm. The particles can be reduced in size in the presence of a surface modifier.

In general, the process for preparing the compositions of the invention would therefore include the steps of milling, such as roller milling, which is described in more detail herein, but may also include other techniques such as GMP methods, e.g. high shear bead milling (Netzsch milling, etc), which allows scale up (including GMP scale up), for instance upon optimization of the process parameters. In general, all these milling processes may be encompassed within the term “grinding medium” used herein.

A general procedure for preparing the particles of this invention comprises
(a) obtaining bedaquiline in micronized form;
(b) adding the micronized bedaquiline to a liquid medium to form a premix/predispersion; and
(c) subjecting the premix to mechanical means in the presence of a grinding medium to reduce the average effective particle size.

Bedaquiline in micronized form is prepared using techniques known in the art. It is preferred that the average effective particle size of the bedaquiline active agent in the predispersion be less than about 100 μm as determined by sieve analysis. Where the average effective particle size of the micronized bedaquiline is greater than about 100 μm, it is preferred that the particles of the bedaquiline compound be reduced in size to less than 100 μm (for example to a size or size range as described herein).

The micronized bedaquiline can then be added to a liquid medium in which it is essentially insoluble to form a predispersion. The concentration of bedaquiline in the liquid medium (weight by weight percentage) can vary widely and depends on the selected surface modifier and other factors. Suitable concentrations of bedaquiline in compositions vary between about 10% to about 30%, e.g. about 10%, 20% or 30% (each % in this paragraph relating to w/v).

The premix can be used directly by subjecting it to mechanical means to reduce the effective average effective particle size in the dispersion to less than 2,000 nm. It is preferred that the premix be used directly when a ball mill is used for attrition. Alternatively, bedaquiline and, optionally, the surface modifier, can be dispersed in the liquid medium using suitable agitation such as, for example, a roller mill, until a homogeneous dispersion is achieved.

The mechanical means applied to reduce the effective average effective particle size of bedaquiline conveniently can take the form of a dispersion mill. Suitable dispersion mills include a ball mill, an attritor/attrition mill, a vibratory mill, a planetary mill, media mills, such as a sand mill and a bead mill. A media mill is preferred due to the relatively shorter milling time required to provide the desired reduction in particle size. The beads preferably are ZrO₂ beads. For instance, for the microparticles, the ideal bead size is about 2 mm.

The grinding media for the particle size reduction step can be selected from rigid media preferably spherical or particulate in form having an average size less than 3 mm and, more preferably, less than 1 mm (as low as 200 μm beads). Such media desirably can provide the particles of the invention with shorter processing times and impart less wear to the milling equipment. Examples of grinding media are ZrO₂ such as 95% ZrO₂ stabilized with magnesia or stabilized with yttrium, zirconium silicate, glass grinding media, polymeric beads, stainless steel, titania, alumina and the like. Preferred grinding media have a density greater than 2.5 g/cm³ and include 95% ZrO₂ stabilized with magnesia and polymeric beads.

The attrition time can vary widely and depends primarily upon the particular mechanical means and processing conditions selected. For rolling mills, processing times of up to two days or longer may be required.

The particles should be reduced in size at a temperature that does not significantly degrade the bedaquiline compound. Processing temperatures of less than 30 to 40° C. are ordinarily preferred. If desired, the processing equipment may be cooled with conventional cooling equipment. The method is conveniently carried out under conditions of ambient temperature and at processing pressures, which are safe and effective for the milling process.

The pharmaceutical compositions according to the present invention contain an aqueous carrier that preferably is pharmaceutically acceptable. Said aqueous carrier comprises sterile water optionally in admixture with other pharmaceutically acceptable ingredients. The latter comprise any ingredients for use in injectable formulations. Such ingredients are optional. These ingredients may be selected from one or more of a suspending agent, a buffer, a pH adjusting agent, a preservative, an isotonizing agent, and the like ingredients. In one embodiment, said ingredients are selected from one or more of a suspending agent, a buffer, a pH adjusting agent, and optionally, a preservative and an isotonizing agent. Particular ingredients may function as two or more of these agents simultaneously, e.g. behave like a preservative and a buffer, or behave like a buffer and an isotonizing agent. Suitable optional buffering agents and pH adjusting agents should be used in amount sufficient to keep the dispersion around neutral, preferably in the pH range of 5.5 to 7.5, for instance around 5.5 to 6.6 (and in an embodiment, it is preferred that the pH is about pH 6). Particular buffers are the salts of week acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl)aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment, the compositions of the invention do not contain a buffering agent. In another embodiment, the compositions of the invention do contain a buffering agent, for instance citric acid (anhydrous) and disodium hydrogen phosphate (anhydrous parenteral)—thus forming a citrate-phosphate buffer—in an embodiment the buffer is present in about 0.5% to 1.5% w/v, e.g. about 0.9% w/v (of which the citric acid is present in about 0.2% to 0.4% w/v, e.g. 0.3%, and the phosphate part is present in about 0.5% to 0.7% w/v, e.g. 0.6%). In an embodiment sodium hydroxide and/or hydrochloric acid may be employed in the compositions of the invention at the outset in order to set the pH to be about pH 5.5 to 6.5 (e.g. about pH 6).

Suitable optional preservatives comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-7-piccolinium chloride, phenylmercuric acetate and thimerosal. Radical scavengers include BHA, BHT, Vitamin E and ascorbyl palmitate, and mixtures thereof. Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA and malic acid. In an embodiment of the invention, the compositions of the invention do not contain a perseverative.

An isotonizing agent or isotonifier may be present to ensure isotonicity of the pharmaceutical compositions of the present invention, and includes sugars such as glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol. Alternatively, sodium chloride, sodium sulfate, or other appropriate inorganic salts may be used to render the solutions isotonic. These isotonifiers can be used alone or in combination. The suspensions conveniently comprise from 0 to 10% (w/v), in particular 0 to 6% of isotonizing agent. Of interest are nonionic isotonifiers, e.g. glucose, as electrolytes may affect colloidal stability. In an embodiment of the invention, the compositions of the invention contain a isotonizing agent or isotonifier, which, in a further embodiment is a nonionic isotonifier, such as a suitable sugar such as mannitol. In another embodiment, the compositions of the invention do not contain an isotonizing agent (also referred to as an osmotic agent).

A desirable feature for a pharmaceutical composition of the invention relates to the ease of administration. The viscosity of the pharmaceutical compositions of the invention should be sufficiently low to allow administration by injection. In particular they should be designed so that they can be taken up easily in a syringe (e.g. from a vial), injected through a fine needle (e.g. a 20 G 1½, 21 G 1½, 22 G 2 or 22 G 1¼ needle) in not too long a time span. In one embodiment the viscosity of the compositions of the invention is below about 75 mPa·s, or below 60 mPa·s. Aqueous suspensions of such viscosity or lower usually meet the above-mentioned criteria.

As indicated herein, in an embodiment, the present invention may therefore concern a pharmaceutical composition for administration by intramuscular or subcutaneous injection, comprising a therapeutically effective amount of bedaquiline, or a pharmaceutically acceptable salt thereof, in the form of a suspension of microparticles, prepared by an aseptic manufacturing process, and comprising:
(a) bedaquiline, or a pharmaceutically acceptable salt thereof, in microparticle form, having a surface modifier adsorbed to the surface thereof; and
(b) a pharmaceutically acceptable aqueous carrier; wherein the bedaquiline active ingredient is suspended,
wherein the bedaquiline (or pharmaceutically acceptable salt thereof) is present, by weight based on the total volume of the composition, from 10% to 30% (w/v) and the surface modifier present from 5% to 15% (w/v), and which comprises at least 30% PEG4000 (or the like) and at least 30% poloxamer (such as poloxamer 338).

The compositions of the invention are suspensions of microparticles, prepared by an aseptic manufacturing process, and as indicated the bedaquiline (or pharmaceutically acceptable salt thereof) and surface modifier (PEG4000 and poloxamer) are present in certain amounts; in certain further embodiments, they are present as following:
bedaquiline (or pharmaceutically acceptable salt thereof) is present from about 15% to 25% (w/v), for instance about 20% (w/v); and/or
the surface modifier is present from about 8% to 12% (w/v), which comprises the PEG4000 to poloxamer ratios described herein

The compositions of the invention have been optimized based on a number of parameters, and the optimized surface modifier quantities are as mentioned above; in certain further embodiments, they may also be present in the following quantities:
the surface modifier comprises between 30% and 70% PEG4000 (or the like) and hence the remainder consists of between 30% and 70% poloxamer;
the surface modifier comprises between 40% and 60% PEG4000, and between 40% and 60% poloxamer
the surface modifier comprises between 45% and 55% PEG4000 (e.g. about 50%), and between 45% and 50% poloxamer (e.g. about 50%)
the surface modifier comprises about 2 to 15% (e.g. 4 to 6%) PEG4000 (w/v), and about 2 to 15% (e.g. 4 to 6%) poloxamer (w/v)
the surface modifier comprises about 5% PEG4000 (w/v), and about 5% poloxamer (w/v)

The compositions of the invention are also indicated as containing a pharmaceutically acceptable carrier, which is described in more detail herein. For instance, the composition of the invention may comprise one or more buffering agents, one or more isotonizing agents (also known as osmotic agents) and one or more preservatives (although in an embodiment, no preservative is present); further the compositions of then invention may contain water for injection (q.s.), and base (e.g. sodium hydroxide; q.s.) and/or acid (q.s.), with the latter two present in order to set the pH at the outset (e.g. to about pH 6).

For instance, the composition of the invention, prepared by an aseptic manufacturing process, may comprise:
(a) bedaquiline, or a pharmaceutically acceptable salt thereof, which is present in an amount between about 180 mg/ml to about 220 mg/ml (e.g. about 200 mg/ml) (and where the weight is based on the active moiety, not the salt component if present);
(b) a surface modifier comprising PEG4000 or the like and a poloxamer (e.g. poloxamer 338), and wherein the PEG4000 or the like is present in an amount between about 40 mg/ml and 60 mg/ml (e.g. about 50 mg/ml), and the poloxmer is present in an amount between about 40 mg/ml and 60 mg/ml (e.g. about 50 mg/ml)
(c) a buffering agent, which is present in about 8 mg/ml to about 10 mg/ml (e.g about 9 mg/ml), for instance, which consists of citric acid (present in about 2 to 4 mg/ml, e.g. about 3 mg/ml) and disodium hydrogen phosphate (present in about 5 to 7 mg/ml, e.g. about 6 mg/ml)
(d) an isotonizing agent (e.g. mannitol), which is present in about 5 mg/ml to about 20 mg/ml, for instance between 10 to 15 mg/ml, e.g. about 13.5 mg/ml); however, in an embodiment an isotonizing agent (e.g. mannitol) is not present, which may be an advantage
(e) a preservative, which may be present in 0 to about 20 mg/ml, but in an embodiment is not present; it may be an advantage that preservative is not needed
(f) water for injection (q.s.), sodium hydroxide and/or hydrochloric acid (both q.s. to adjust pH as described herein)

To the suspensions may optionally be added an amount of acid or base to bring the pH to a value of about pH 7. Suitable acids or bases are any of those that are physiologically acceptable, e.g. HCl, HBr, sulfuric acid, alkali metal hydroxides such as NaOH. In an embodiment, such acid or base need not be added to the compositions of the invention.

The administration of bedaquiline (or pharmaceutically acceptable salt thereof) as in the present invention may suffice to treat a pathogenic mycobacterial infection although in a number of cases it may be recommendable to co-administer other anti-TB drugs.

In certain instances, the treatment of a pathogenic mycobacterial infection may be limited to only the administration of a composition of bedaquiline (and/or its metabolite thereof) in accordance with this invention, i.e. as monotherapy without co-administration of further anti-TB drugs. This option may be recommended, for example, for certain mycobacterial infections where a low concentration of the active ingredient may treat the bacteria (e.g. for latent/dormant TB or for Mycobacterium leprae).

In a further aspect the present invention relates to the use of a pharmaceutical composition comprising an effective amount of bedaquiline or a pharmaceutically acceptable salt thereof, in accordance with the present invention, for the manufacture of a medicament for maintenance therapy of a subject being infected with a pathogenic mycobacterial infection, wherein the composition is administered or is to be administered intermittently at a time interval that is in the range of one week to one year, or one week to two years.

Thus in a further aspect, the present invention provides a method for the long term treatment of a patient being infected with a pathogenic mycobacterial infection, said method comprising
(i) the treatment of said patient with a combination of anti-TB drugs; followed by
(ii) the intermittent administration of a pharmaceutical composition comprising an effective amount of bedaquiline or a pharmaceutically acceptable salt thereof, in accordance with the present invention, wherein the composition is administered at a time interval of at least one week.

Where, the treatment is directed towards Mycobacterium leprae, then again the treatment regime might be given as monotherapy or in combination with existing drugs useful for the treatment of Mycobacterium leprae (e.g. rifapentin). The composition of the invention might be administered by injection once, or up to three times, e.g. as monthly intervals. Advantages are associated with compliance, no resistance by avoiding dapsone, no stigma by avoiding clofazimine.

The present invention also concerns a pharmaceutical composition as described hereinbefore for use as a medicament in the treatment or prophylaxis of a pathogenic mycobacterial infection.

In addition, the present invention concerns the use of a pharmaceutical composition as described herein for the preparation of a medicament for the prophylaxis or treatment of a pathogenic mycobacterial infection.

The present invention further concerns a method of treating a subject infected with a pathogenic mycobacterial infection, said method comprising the administration of a therapeutically effective amount of a pharmaceutical composition as described herein.

As used herein, the word “substantially” does not exclude “completely” e.g. a composition which is “substantially free” from Y may be completely free from Y. Where necessary, the word “substantially” may be omitted from the definition of the invention. The term “about” in connection with a numerical value is meant to have its usual meaning in the context of the numerical value. Where necessary the word “about” may be replaced by the numerical value ±10%, or ±5%, or ±2%, or ±1%.

All documents cited herein are incorporated by reference in their entirety.

The following examples are intended to illustrate the present invention and should not be construed as limiting the invention thereto.

### EXAMPLES

### Process Example: Preparation of Micro-Suspensions

The active ingredient bedaquiline may be used as such or may be converted into a pharmaceutically acceptable salt thereof, such as a fumarate salt (for example the form used in the marketed product Sirturo®). Where referred to herein, bedaquiline is used in its non-salt form unless otherwise specified.

The process of the preparation of compositions of the invention is by an aseptic manufacturing process, where the bedaquiline is γ-irradiated. The bedaquiline compositions may be prepared in accordance with the following:
Preparation of 200 and 100 mg/mL micro-suspensions.
Materials used:
Zirconium beads 0.5 mm (to aid process)
Sterile water for injection (Viaflo)
Bedaquiline (not milled/ground)—this API is γ-irradiated
Surface modifier (for instance, including PEG4000 (or the like) (50 mg/ml) and poloxamer
(50 mg/ml))—excipient(s)
Zirconium beads 2 mm (to aid process)
Mannitol (parenteral)—an excipient

Glass bottles and ZrO₂ beads (either 0.5 mm or 2 mm, depending on the desired micro-suspensions), used as the milling media, were sterilized in an autoclave. The drug substance (quantity depending on the formulation to be prepared; see e.g. formulation/suspension below) was put into the glass bottle as well as a solution of surface modifier (PEG4000 (or the like) (50 mg/ml) and poloxamer (50 mg/ml)) in water (quantity depending on the concentration required/desired; see e.g. formulation/suspension below) for injection. ZrO₂-beads with an average particle size of 500 μm or 2 mm (depending on whether a micro- or nano-suspension is required/desired) were added. The bottle was placed on a roller mill. The suspension was micronized at 100 rpm for a period of time up to 72 hours. For instance, micronizing may be performed at 100 rpm for a period of 3 hours (or up to 3 hours). At the end of the milling process the concentrated micro-suspension was removed with a syringe and filled into vials. The resulting formulations (based on the micro-suspension) are described herein. Determination of the concentration was done by HPLC/UV. If needed, a dilution was made to a final concentration of 200 mg/ml of active ingredient bedaquiline. The resulting suspension was shielded from light.

In general, the process for preparing the compositions of the invention would therefore include the steps of milling, such as roller milling, which is described in more detail herein, but may also include other techniques such as GMP methods, e.g. high shear bead milling (Netzsch milling, etc), which allows scale up (including GMP scale up), for instance upon optimization of the process parameters.

Such formulations were (and will be) dosed intramuscular and subcutaneous in animals for PK study to investigate a possible long-acting effect (e.g. in treatment of tuberculosis, for instance latent tuberculosis, or even for the treatment of leprosy).

Physical stability of the suspensions will be followed up by measuring particle size after different storage conditions.

Certain embodiments of the formulation(s) have the following features:
Micro-suspension by using 2 mm Zr beads
Milling at 200 mg/mL (otherwise the concentration may be too high, e.g. with 300 mg/ml)
Longer milling would result in nano-suspension
A suitable surface modifier, for instance selected based on physical stability, e.g. a surface modifier or wetting agent as described herein

### Reference Examples of Bedaquiline Micro-Suspensions

200 mg/ml Micro-Suspension Referred to Herein as Reference Example A (without Buffer) and Reference Examples B and C (with Buffer)

### Reference Example A

| | mg/ml |
|---|---|
| Bedaquiline | 200 |
| TPGS | 50 |
| Mannitol | 50 |
| Sterile water for injection | q.s. |

### Particle Size Distribution (PSD)

| | Storage | | | |
|---|---|---|---|---|
| Storage | temperature | | | |
| time | (°C) | D10 (μm) | D50 (μm) | D90 (μm) |
| 0 | | 0.820 | 1.99 | 4.96 |
| 1 month | 25 °C | 0.704 | 1.59 | 3.64 |

The PSD measurements after 1 month indicate that formulation remains relatively stable.

### Stability Test Using HPLC:

An HPLC test method was used to determine how stable the long acting injectable formulation of Reference Example A is. The purpose was to measure the amount of bedaquiline relative to two known degradants after certain periods of time at room temperature.

HPLC Procedure: Column—ProntoSIL 120-3-C18 SH, 100 mm length×3.0 mm i.d., 3 μm particle size, or equivalent; column temperature 35° C.; auto-sampler temperature 5° C.; Flow rate 0.5 mL/min; Detection UV; Wavelength 230 nm; Data Collection Time 50 minutes; Analysis Run Time 60 minutes; Injection volume 10 μl; Mobile Phase A is 0.03 M Hydrochloric Acid in Water; Mobile Phase B is Methanol/Acetonitrile/2-Propanol—45/45/10 (v/v/v).

| Time (at room | Degradant A | Degradant B | Bedaquiline |
|---|---|---|---|
| temperature) | (%, w/w) | (%, w/w) | (%, w/w) |
| Time zero | 0.49 | 0.05 | 102.5 |
| 3 months | 0.50 | 0.06 | 102.3 |
| 6 months | 0.50 | 0.08 | 103.2 |

The HPLC purity test shows that the formulation of Reference Example A is relatively stable for a long period (given that the relative amounts of degradants and bedaquiline remained stable).

### Reference Example B and C

| | Reference | Reference |
|---|---|---|
| | Example B mg/ml | Example C mg/ml |
| | (where applicable) | (where applicable) |
| Bedaquiline | 200 | 200 |
| TPGS | 50 | 50 |
| Buffer - citrate-phosphate | 0.01N (pH 6) | 0.05N (pH 6) |
| Mannitol | 50 | 50 |
| Sterile water for injection | q.s. | q.s. |

### Particle Size Distribution (PSD)

| Reference | | | | |
|---|---|---|---|---|
| Example | Conditions | D10 (μm) | D50 (μm) | D90 (μm) |
| B | After 3 hrs | 0.856 | 2.28 | 5.38 |
| | milling | | | |
| C | After 3 hrs | 0.969 | 2.54 | 6.29 |
| | milling | | | |
| B | After | 1.13 | 2.29 | 4.72 |
| | autoclaving | | | |
| C | After | 1.15 | 2.37 | 5.10 |
| | autoclaving | | | |
| B | After 1 month at | 1.13 | 2.29 | 4.68 |
| | 25 °C | | | |
| C | After 1 month at | 1.16 | 2.36 | 4.96 |
| | 25 °C | | | |
| B | After | 1.12 | 2.27 | 4.65 |
| | autoclaving and | | | |
| | 1 month at 5 °C | | | |
| C | After | 1.15 | 2.35 | 4.98 |
| | autoclaving and | | | |
| | 1 month at 5 °C | | | |

The PSD for these formulations under various conditions (including after autoclaving) indicate that the formulations remain relatively stable.

### Reference Example D

PEG4000 (or polyethylene glycol 4000) is used, which can be supplied from Clariant GmbH. PEG4000 is a hydrophilic agent that can be used to increase the viscosity of the suspending vehicle and can act as a suspending agent.

| | mg/ml |
|---|---|
| Bedaquiline | 200 |
| PEG4000 | 50 |
| TPGS | 5 |
| Buffer - citrate-phosphate | 0.01N (pH 6) |
| Mannitol | 50 |
| Sterile water for injection | q.s. |

In this case a buffer was added to avoid a drop in pH.

### Particle Size Distribution (PSD)

| Conditions | D10 (μm) | D50 (μm) | D90 (μm) |
|---|---|---|---|
| Before autoclaving | 1.27 | 3.29 | 10.4 |
| After autoclaving | 1.22 | 2.74 | 6.72 |

The PSD of the micro-suspension of Reference Example D shows that the formulation remains relatively stable even after autoclaving.

The approximate cloud point of the formulation of Reference Example D was calculated to be about 105 to 110° C.

The autoclaving of the micro-suspension of Reference Example D was conducted in a Systec autoclave (VX/VE series), where the present parameters are:
Sterilization temperature: 121° C. (above the calculated cloud point)
Sterilization time: 15 minutes
Unloading temperature: 80° C.

A typical autoclaving cycle—the steam generator builds up the required steam pressure and the steam flows into the sterilization chamber, after the sterilization temperature has been reached, it then remains constant for the duration of the sterilization period, and after the period has elapsed the cycles with the optional built-in cooling apparatus are cooled down until the unloading temperature has been reached.

### Reference Example E1 and E2

| | Reference Example | Reference Example |
|---|---|---|
| | E1 | E2 |
| API (mg/ml) | Bedaquiline 200 mg/ml | Bedaquiline 200 mg/ml |
| Surface Modifier | P338 (20 mg/ml) | P188 (40 mg/ml) |
| (mg/ml) | | |
| Buffer | Citrate-phosphate | Citrate |
| Isotonifying agent | Mannitol | Mannitol |
| Sterile water for | q.s. | q.s. |
| injection | | |

Buffer was added to avoid a drop in pH.

### Particle Size Distribution (PSD)

| | Reference Example E1 | Reference Example E2 |
|---|---|---|
| D10 (μm) | 0.844 | 1.03 |
| D50 (μm) | 3.08 | 3.44 |
| D90 (μm) | 8.6 | 10.0 |

### Objective Stability Testing Criteria

Various formulations/suspensions/concepts were tested, each of which underwent a specific sterilization technique:
   Autoclaving (see e.g. methods above)
   γ-irradiation of the suspensions (with such methods described above, in the background section for example)
   Aseptic manufacturing (with γ-irradiated API)
While each type of sterilization technique may have specific advantages, and certain formulations may have advantages in that they may be amenable to or tolerant to specific sterilization techniques, the formulations (or suspensions/concepts) may also be tested with regards to their stability as follows:

### Chemical Stability

Assay/purity by UHPLC
Aldehyde content by UHPLC
VitE TPGS content measurement by UHPL (for the corresponding formulations containing it)
Peroxide content by UPLC/MS for γ-irradiated suspensions
pH

### Physical Stability

Appearance/Injectability/Re-suspendability
PSD
XRD

For all the stability testing, this can be done:
after 1, 3 and 6M for samples stored at certain temperatures and at certain relative humidity, for instance, 30° C./75% RH and 40° C./75% RH
and after 28 days for samples stored at 60° C. (assay/purity) and 50° C. (PSD and XRD)

### EXAMPLES

Depending on the sterilization techniques, the formulation/suspension can have different properties, for instance different chemical and physical stability properties, and the following examples where prepared and tested.

### Example 1—a Micro-Suspension of the Invention

### Example 1A Formulation

| | mg/ml (where applicable) |
|---|---|
| Bedaquiline | 200 |
| PEG4000 | 50 |
| Poloxamer 338 | 50 |
| Citric acid anhydrous | 3.068 |
| Disodium hydrogen phosphate anhydrous | 6.076 |
| Sterile water for injection | q.s. |
| Sodium hydroxide | q.s. to adjust to pH 6 |
| Conc. hydrochloric acid | q.s. to adjust to pH 6 |

In this case a buffer was added to avoid a drop in pH; and buffer was, in this case a mixture of citric acid and a phosphate (a sodium phosphate; 50 mM phosphate-citrate buffer); additionally, in order to adjust the pH at the outset sodium hydroxide and/or hydrochloric acid may be employed. The target pH is pH 6.

It does not contain an isotonizing agent (also referred to as an osmotic agent), such as mannitol This formulation Example 1A was prepared using aseptic manufacturing (with γ-irradiated API)—so specifically, the API product (bedaquiline) is γ-irradiated, and the suspension is then made up using aseptic manufacturing techniques, for instance using sterilized equipment or other techniques to ensure that the end formulation/suspension produced is sufficiently absent of bacteria.

As mentioned above, the formulations/suspensions may also be autoclaved or the entire end formulation/suspension may be γ-irradiated. However, the formulation Example 1A has chemical stability and/or physical stability advantages, for instance compared to known bedaquiline long-acting formulations/suspensions, or compared to formulations/suspensions prepared by other methods, e.g. autoclaving, or γ-irradiation of entire formulation/suspension.

### Stability Testing Results

### For Formulations of the Invention

Chemical Stability—this was Tested at 30° C./75% RH and 40° C./75% RH
Assay/purity by UHPLC

| | | | RRT 0.20 | RRT 1.36 |
|---|---|---|---|---|
| Formulation | Condition | Bedaquiline | (%) | (%) |
| Example 1A | T0 | 101.0 | <0.05 | <0.05 |
| | T = 1 M 30 °C | 100.5 | <0.05 | <0.05 |
| | T = 1 M 40 °C | 101.0 | <0.05 | <0.05 |
| | T = 28 D 60 °C | 100.6 | <0.05 | 0.11 |
| | T = 3 M 30 °C | 104.7 | <0.05 | <0.05 |
| | T = 3 M 40 °C | 101.8 | <0.05 | <0.05 |
| | T = 6 M 30 °C | 101.3 | <0.05 | <0.05 |
| | T = 6 M 40 °C | 100.6 | <0.05 | <0.05 |

where e.g. T=1M 30° C. means, time period of 1 month at a temperature of 30° C., 28D=28 days, etc
Aldehyde (formaldehyde, acetaldehyde, propionaldehyde) content by UHPLC

| | | Formal- | Acetal- | Propion- | Sum of |
|---|---|---|---|---|---|
| Formula- | | dehyde | dehyde | aldehyde | aldehydes |
| tion | Condition | (ppm) | (ppm) | (ppm) | (ppm) |
| Example | T0 | 3 | 14 | 36 | 53 |
| 1A | T = 1 M | 4 | 13 | 30 | 47 |
| | 30 °C | | | | |
| | T = 1 M | 5 | 14 | 30 | 49 |
| | 40 °C | | | | |
| | T = 3 M | 5 | 13 | 29 | 47 |
| | 30 °C | | | | |
| | T = 3 M | 6 | 14 | 25 | 45 |
| | 40 °C | | | | |
| | T = 6 M | 5 | 14 | 25 | 44 |
| | 30 °C | | | | |
| | T = 6 M | 8 | 14 | 17 | 39 |
| | 40 °C | | | | |

VitE TPGS content measurement by UHPL is not applicable for this formulation/suspension
pH

| Formulation | Condition | pH |
|---|---|---|
| Example 1A | T0 | 6.2 |
| | T = 1 M 30 °C | 6.2 |
| | T = 1 M 40 °C | 6.2 |
| | T = 1 M 50 °C | 6.2 |
| | T = 3 M 30 °C | 6.2 |
| | T = 3 M 40 °C | 6.2 |
| | T = 6 M 5 °C | 6.2 |
| | T = 6 M 30 °C | 6.2 |
| | T = 6 M 40 °C | 6.1 |

### Physical Stability at 30° C./75% RH and 40° C./75% RH

Appearance/Injectability/Re-suspendability

| Formulation | Condition | Pass/Fail | Remark |
|---|---|---|---|
| Example 1A | T0 | Pass | |
| | T = 1 M 30 °C | Pass | 3/3 vials - small soft white |
| | | | aggregates |
| | T = 1 M 40 °C | Pass | 3/3 vials - small soft white |
| | | | aggregates |
| | T = 1 M 50 °C | Pass | 3/3 vials - small soft white |
| | | | aggregates |
| | T = 3 M 30 °C | Pass | |
| | T = 3 M 40 °C | Pass | |
| | T = 6 M 5 °C | Pass | |
| | T = 6 M 30 °C | Pass | |

PSD

| Formulation | Condition | D10 (μm) | D50 (μm) | D90 (μm) |
|---|---|---|---|---|
| Example 1A | T0 | 0.7 | 2.5 | 6.2 |
| | T = 1 M 30 °C | 0.8 | 2.5 | 5.8 |
| | T = 1 M 40 °C | 0.8 | 2.5 | 5.7 |
| | T = 1 M 50 °C | 0.9 | 2.5 | 5.7 |
| | T = 3 M 30 °C | 0.8 | 2.5 | 5.9 |
| | T = 3 M 40 °C | 0.8 | 2.5 | 6.0 |
| | T = 6 M 5 °C | 0.8 | 2.5 | 5.7 |
| | T = 6 M 30 °C | 0.7 | 2.4 | 6.2 |
| | T = 6 M 40 °C | 0.9 | 2.6 | 6.0 |

XRD

| Formulation | Condition | XRD |
|---|---|---|
| Example 1A | T0, T = 1 M, T = 3 M | Bedaquiline in preferred |
| | | crystalline form |

### Conclusions—Chemical Stability

The purity assay of formulation Example 1A remained stable up to 6 months; no impurities above the reporting threshold of 0.05% were detected; after 28 days at 60° C. an impurity X was formed (~0.10%)—where impurity X had a RRT of 1.36
The aldehyde content was measured and:—for formulation Example 1A minimal or no changes were observed in the formaldehyde, acetaldehyde, propionaldehyde contents
Minimal or no changes were observed in pH value

### Conclusions—Physical Stability

Some soft aggregates were noticed after 1M storage at 30° C., 40° C., and 50° C. for formulation Example 1A (however these were not seen at the 3M timepoints, nor at the 6M timepoints measured at certain temperatures)
No changes/no significant changes were noticed in the particle size (per the PSD—particle size distribution)
XRD—x-ray diffraction—the preferred crystalline form was observed at all time times (the crystalline form of bedaquiline described herein)

In summary, the formulations of the invention/Example 1A may have chemical and/or physical stability advantages based on any of the criteria measured herein (e.g. purity analysis, aldehyde content, pH, appearance/re-suspendability/injectability, PSD and/or XRD)

### For Other Concepts

### Other Concepts:

### Concept A Formulation—Prepared by Autoclaving:

| | mg/ml (where applicable) |
|---|---|
| Bedaquiline | 200 |
| PEG4000 | 50 |
| Poloxamer 338 | 50 |
| Mannitol | TBD |
| Citric acid anhydrous | TBD |
| Disodium hydrogen phosphate anhydrous | TBD |
| Sterile water for injection | q.s. |
| Sodium hydroxide | q.s. to adjust to pH 6 |
| Conc. hydrochloric acid | q.s. to adjust to pH 6 |

In this case a buffer may be added to avoid a drop in pH; and buffer was, in this case a mixture of citric acid and a phosphate (a sodium phosphate); additionally, in order to adjust the pH at the outset sodium hydroxide and/or hydrochloric acid may be employed; the amount of buffer can be determined—for instance 50 mM phosphate-citrate buffer is employed with the target pH 6.

The amount of mannitol (isotonizing agent, or which can also be termed an osmotic agent) can also be determined, e.g. based on the quantities used in Example 1A/1B above

This formulation Concept A was prepared by autoclaving (see here for methods).

### Concept B Formulation—Prepared by γ-Irradiation of Final Formulation/Suspension:

| | mg/ml (where applicable) |
|---|---|
| Bedaquiline | 200 |
| PEG4000 | 50 |
| Vit E TPGS | 10 |
| Mannitol | TBD |
| Citric acid anhydrous | TBD |
| Disodium hydrogen phosphate anhydrous | TBD |
| Sterile water for injection | q.s. |
| Sodium hydroxide | q.s. to adjust to pH 6 |
| Conc. hydrochloric acid | q.s. to adjust to pH 6 |

In this case a buffer may be added to avoid a drop in pH; and buffer was, in this case a mixture of citric acid and a phosphate (a sodium phosphate); additionally, in order to adjust the pH at the outset sodium hydroxide and/or hydrochloric acid may be employed; the amount of buffer can be determined—for instance 50 mM phosphate-citrate buffer is employed with the target pH 6.

The amount of mannitol (isotonizing agent, or which can also be termed an osmotic agent) can also be determined, e.g. based on the quantities used in Example 1A/1B above

This formulation Concept B was prepared by γ-irradiation of the final formulation/suspension

### Concept C Formulation—Prepared by γ-Irradiation of Final Formulation/Suspension:

| | mg/ml (where applicable) |
|---|---|
| Bedaquiline | 200 |
| PEG4000 | 50 |
| Vit E TPGS | 20 |
| Mannitol | TBD |
| Citric acid anhydrous | TBD |
| Disodium hydrogen phosphate anhydrous | TBD |
| Sterile water for injection | q.s. |
| Sodium hydroxide | q.s. to adjust to pH 6 |
| Conc. hydrochloric acid | q.s. to adjust to pH 6 |

In this case a buffer may be added to avoid a drop in pH; and buffer was, in this case a mixture of citric acid and a phosphate (a sodium phosphate); additionally, in order to adjust the pH at the outset sodium hydroxide and/or hydrochloric acid may be employed; the amount of buffer can be determined—for instance 50 mM phosphate-citrate buffer is employed with the target pH 6.

The amount of mannitol (isotonizing agent, or which can also be termed an osmotic agent) can also be determined, e.g. based on the quantities used in Example 1A/1B above

This formulation Concept C was prepared by γ-irradiation of the final formulation/suspension.

Concept D Formulation—Reared by q-Irradiation of Final Formulation/Suspension:

| | mg/ml (where applicable) |
|---|---|
| Bedaquiline | 200 |
| PEG4000 | 50 |
| Poloxamer 338 | 50 |
| Mannitol | TBD |
| Citric acid anhydrous | TBD |
| Disodium hydrogen phosphate anhydrous | TBD |
| Sterile water for injection | q.s. |
| Sodium hydroxide | q.s. to adjust to pH 6 |
| Conc. hydrochloric acid | q.s. to adjust to pH 6 |

In this case a buffer may be added to avoid a drop in pH; and buffer was, in this case a mixture of citric acid and a phosphate (a sodium phosphate); additionally, in order to adjust the pH at the outset sodium hydroxide and/or hydrochloric acid may be employed; the amount of buffer can be determined—for instance 50 mM phosphate-citrate buffer is employed with the target pH 6.

The amount of mannitol (isotonizing agent, or which can also be termed an osmotic agent) can also be determined, e.g. based on the quantities used in Example 1A/1B above This formulation Concept D was prepared by γ-irradiation of the final formulation/suspension.

### In the Chemical Stability and Physical Stability Testing Criteria, the Following was Noted:

Concept A failed the physical stability test for appearance/injectability/re-suspendability:
   at 1M and 3M at 40° C. because of extraction challenges (and after 3M, caking at the bottom of the vial was detected)
   at 3M at 25° C. due to the needle clogging during extraction
Concepts B and C failed as the amount of Vit E TPGS decreased immediately after γ-irradiation of the formulation/suspension by ~20%—only 75-80% of the Vit E TPGS remained (although a further decrease was not measured after this initial drop)
Concept D failed in the physical stability testing, on at least appearance, as even at T0, formulation caking was already seen at the bottom of the flasks

### Biological Example: Pharmacokinetic Studies

Example 1A was tested in rats, with the following results.

| | Intramuscular (IM) delivery - 40 mg/kg (0.2 mL/kg) | |
|---|---|---|
| | Example 1A | |
| T max (h)* | 24 | (7-48) |
| Cmax (ng/mL) | 120 | (17%) |
| AUC_{34 days} | 32300 | (12%) |
| (ng*h/mL) | | |
| F % | 36 | |
| AUC_{6 months} | 67200 | (8%) |
| (ng*h/mL) | | |
| F % | 76 | |
| % of the dose at | 3.9 | |
| the injection at 6 | | |
| mths | | |

*median (min-max)

Example 1A was tested in dogs, with the following results.

Example 1A - 40 mg/kg (0.2 mL/kg)

| | Individual Data | | | Mean | CV (%) |
|---|---|---|---|---|---|
| Cmax | 373 | 495 | 290 | 386 | 27 |
| (ng/mL) | | | | | |
| T max (h)* | 7 | 7 | 7 | 7 (7-7) | |
| AUC_{6 months} | 181563 | 189100 | 92978 | 154547 | 35 |
| (ng*h/mL) | | | | | |

*median (min-max)

1. A pharmaceutical composition for administration by intramuscular or subcutaneous injection, comprising a therapeutically effective amount of bedaquiline, or a pharmaceutically acceptable salt thereof, in the form of a suspension of microparticles, prepared by an aseptic manufacturing process, and comprising:
(a) bedaquiline, or a pharmaceutically acceptable salt thereof, in microparticle form, which is present, by weight based on the total volume of the composition, from 10% to 30% (w/v);
(b) a surface modifier, present from 5% to 15% (w/v), and which comprises at least 30% PEG4000 (or the like) and at least 30% poloxamer (such as poloxamer 338); and
(c) a pharmaceutically acceptable aqueous carrier.

2. A pharmaceutical composition for administration by intramuscular or subcutaneous injection, comprising a therapeutically effective amount of bedaquiline, or a pharmaceutically acceptable salt thereof, in the form of a suspension of microparticles, prepared by an aseptic manufacturing process, and comprising:
(a) bedaquiline, or a pharmaceutically acceptable salt thereof, in microparticle form, having a surface modifier adsorbed to the surface thereof; and
(b) a pharmaceutically acceptable aqueous carrier; wherein the bedaquiline active ingredient is suspended,
wherein the bedaquiline (or pharmaceutically acceptable salt thereof) is present, by weight based on the total volume of the composition, from 10% to 30% (w/v) and the surface modifier present from 5% to 15% (w/v), and which comprises at least 30% PEG4000 (or the like) and at least 30% poloxamer (such as poloxamer 338).

3. A pharmaceutical composition according to claim 1, wherein bedaquiline is in its non-salt or free form or in the form of a fumarate salt.

4. A pharmaceutical composition according to claim 1, wherein the bedaquiline (or pharmaceutically acceptable salt thereof) is present from about 15% to 25% (w/v), for instance about 20% (w/v).

5. A pharmaceutical composition according to claim 1, wherein the surface modifier is present from about 8% to 12% (w/v), which comprises comprises about 5% PEG4000 (w/v), and about 5% poloxamer (w/v).

6. A pharmaceutical composition according to claim 1, wherein the average effective particle size of the bedaquiline, or a pharmaceutically acceptable salt thereof, micro-particles is below about 50 μm.

7. A pharmaceutical composition according to claim 1, prepared by an aseptic manufacturing process, and comprising:
(a) bedaquiline, or a pharmaceutically acceptable salt thereof, which is present in an amount between about 180 mg/ml to about 220 mg
(b) a surface modifier comprising PEG4000 or the like and a poloxamer (e.g. poloxamer 338), and wherein the PEG4000 or the like is present in an amount between about 40 mg/ml and 60 mg/ml, and the poloxmer is present in an amount between about 40 mg/ml and 60 mg/ml
(c) a buffering agent, which is present in about 8 mg/ml to about 10 mg/ml, for instance, which consists of citric acid (present in about 2 to 4 mg/ml) and disodium hydrogen phosphate (present in about 5 to 7 mg/ml)
(d) water for injection (q.s.), sodium hydroxide and/or hydrochloric acid (both q.s. to adjust pH as described herein).

8. A method for the treatment of a pathogenic mycobacterial infection comprising administering a pharmaceutical composition as defined in claim 1 to a subject in need thereof.

9. The method of claim 8, wherein the pharmaceutical composition is for the long-term treatment of Mycobacterium tuberculosis (such as the latent/dormant form) or Mycobacterium leprae.

10. The method according to claim 8, wherein the pharmaceutical composition is for administration by intramuscular or subcutaneous injection; wherein the pharmaceutical composition is administered intermittently at a time interval of one week to two years.

11. The method according to claim 8, wherein the pharmaceutical composition is administered at an interval of at least one month to one year.

12. The method according to claim 8, wherein the pharmaceutical composition is administered at a time interval that is in the range of one week to one month, or in the range of one month to three months, or in the range of three months to six months, or in the range of six months to twelve months, or in the range of 12 months to 24 months.

13. The method according to claim 8, wherein the pharmaceutical composition is administered once every two weeks, or once every month, or once every three months.

14. A process for preparing a pharmaceutical composition as defined in claim 1, comprising
(a) obtaining bedaquiline, or a pharmaceutically acceptable salt thereof, in micronized form;
(b) adding the micronized bedaquiline, or a pharmaceutically acceptable salt thereof, to a liquid medium to form a premix/predispersion; and
(c) subjecting the premix to mechanical means in the presence of a grinding medium to reduce the average effective particle size.
